(19) United States
(12) Patent Application Publication
(10) Pub. No.: US 2017/0173298 A1
(43) Pub. Date: Jun. 22, 2017

(54) Using Audio to Alter Respiratory Rates

(71) Applicant: Plantronics, Inc., Santa Cruz, CA (US)

(72) Inventors: Evan Harris Benway, Santa Cruz, CA (US); Jacob T Meyberg, Santa Cruz, CA (US); Barry L Margerum, Santa Cruz, CA (US)

(73) Assignee: Plantronics, Inc., Santa Cruz, CA (US)

(21) Appl. No.: 14/971,114

(22) Filed: Dec. 16, 2015

(51) Int. Cl. A61M 21/02; A61B 5/0205

(52) U.S. Cl. CPC A61M 21/02; A61B 5/0205; A61M 2021/0027

(57) ABSTRACT

Apparatus having corresponding methods and computer-readable media comprise a controller configured to i) determine a desired respiratory rate of a person, and ii) generate an audio signal having a periodic component, wherein a rate of the periodic component is based on the desired respiratory rate; and a speaker configured to produce sound based on the audio signal.

### FIELD

The present disclosure relates generally to the field of audio processing. More particularly, the present disclosure relates to the use of audio to promote wellness.

### BACKGROUND

Workplace wellness is an important concern and cost center for employers. Stress and emotional distress can notably decrease employee satisfaction, wellness and productivity. Therefore many companies are looking for solutions. Mindful breathing has a demonstrable impact on physical and emotional wellness. Mindfulness programs such as Search Inside Yourself are on the rise. However, all of these programs rely on an individual's motivation to improve his or her physical or emotional wellness.

### SUMMARY

In general, in one aspect, an embodiment features an apparatus comprising: a controller configured to i) determine a desired respiratory rate of a person, and ii) generate an audio signal having a periodic component, wherein a rate of the periodic component is based on the desired respiratory rate; and a speaker configured to produce sound based on the audio signal.

Embodiments of the apparatus may include one or more of the following features. In some embodiments, the controller is further configured to determine a current respiratory rate of the person. In some embodiments, the controller is further configured to determine the desired respiratory rate based on the current respiratory rate. Some embodiments comprise a sensor configured to produce a signal, wherein the signal represents a biometric of the person; wherein the controller is further configured to determine the current respiratory rate of the person based on the signal. In some embodiments, the sensor is wearable. Some embodiments comprise a user interface configured to capture user input of the person; wherein the controller is further configured to determine the current respiratory rate of the person based on the user input. In some embodiments, the controller is further configured to iii) determine a current respiratory rate of a further person, and iv) determine the desired respiratory rate based on the current respiratory rate of the person and the current respiratory rate of the further person.

In general, in one aspect, an embodiment features a method comprising: determining a desired respiratory rate of a person, and generating a sound having a periodic component, wherein a rate of the periodic component is based on the desired respiratory rate.

Embodiments of the method may include one or more of the following features, Some embodiments comprise determining a current respiratory rate of the person. Some embodiments comprise determining the desired respiratory rate based on the current respiratory rate. Some embodiments comprise determining a biometric of the person; and determining the current respiratory rate of the person based on the biometric. Some embodiments comprise capturing user input of the person; and determining the current respiratory rate of the person based on the user input. Some embodiments comprise determining a current respiratory rate of a further person, and determining the desired respiratory rate based on the current respiratory rate of the person and the current respiratory rate of the further person.

In general, in one aspect, an embodiment features computer-readable media embodying instructions executable by a computer to perform functions comprising: determining a desired respiratory rate of a person, and generating a sound having a periodic component, responsive to determining the current respiratory rate, wherein a rate of the periodic component is based on the desired respiratory rate.

Embodiments of the computer-readable media may include one or more of the following features. In some embodiments, the functions further comprise: determining a current respiratory rate of the person. In some embodiments, the functions further comprise: determining the desired respiratory rate based on the current respiratory rate. In some embodiments, the functions further comprise: receiving a biometric of the person; and determining the current respiratory rate of the person based on the biometric. In some embodiments, the functions further comprise: capturing user input of the person; and determining the current respiratory rate of the person based on the user input. In some embodiments, the functions further comprise: determining a current respiratory rate of a further person, and determining the desired respiratory rate based on the current respiratory rate of the person and the current respiratory rate of the further person.

The details of one or more implementations are set forth in the accompanying drawings and the description below. Other features will be apparent from the description and drawings, and from the claims.

### DESCRIPTION OF DRAWINGS

FIG. 1 shows elements of an audio wellness system for one user according to one embodiment.

FIG. 2 shows elements of an audio wellness system for multiple users according to one embodiment.

FIG. 3 shows elements of a headset according to one embodiment.

FIG. 4 shows a process for the audio wellness systems of FIGS. 1 and 2 according to one embodiment.

The leading digit(s) of each reference numeral used in this specification indicates the number of the drawing in which the reference numeral first appears.

### DETAILED DESCRIPTION

Embodiments of the present disclosure provide audio to alter the respiratory rate of one or more listeners. The audio comprises a periodic component having a rate corresponding to a desired respiratory rate. The audio may include nature sounds, pink noise, brown noise, and the like. The audio may be generated responsive to determining the current respiratory rate of a listener. The desired respiratory rate may be determined based on the current respiratory rate of one or more listeners. The current respiratory rate of the listener may be based on one or more biometrics of the listener. The biometrics may include a moisture metric, a heart rate metric, a sound metric, a galvanic skin response metric, a gas metric, a motion metric, and the like. The current respiratory rate may be determined based on user input.

The audio may be continuous. The audio may be generated only responsive to the current respiratory rate being greater than a reference respiratory rate. In that case, the desired respiratory rate may be less than the current respiratory rate. The audio may be generated only responsive to the current respiratory rate being less than a reference respiratory rate. In that case the desired respiratory rate is greater than the current respiratory rate. The audio may be generated only responsive to the current respiratory rate being outside of a desired range. The audio may include an audible marker to indicate a change in the desired respiratory rate.

A user interface may display a visual indication of the current respiratory rate, the desired respiratory rate, the difference between these rates, and the like. Other features are contemplated as well.

In some cases, the user may be consciously aware of the oscillation in the sound pattern and may be given an audio cue such as a brief bell tone to signify that the oscillation pattern is going to change and that the user should consciously follow it. In other cases, the user may not consciously be aware of the oscillation, or at least may not be given a cue indicating that the oscillation of the sound pattern is changing. In such cases, the user's breathing may follow the oscillation of the sound pattern without the user being consciously aware of the fact. This phenomenon, known as “entrainment,” occurs when two oscillations tend to fall into synchrony. One example is the tendency in many cultures for applause to fall into unison, especially at the end of a performance. With entrainment the user does not need to understand how the system works or consciously apply himself to the method. Simply operating the system is enough to bring about the desired reduction in the user's respiratory rate.

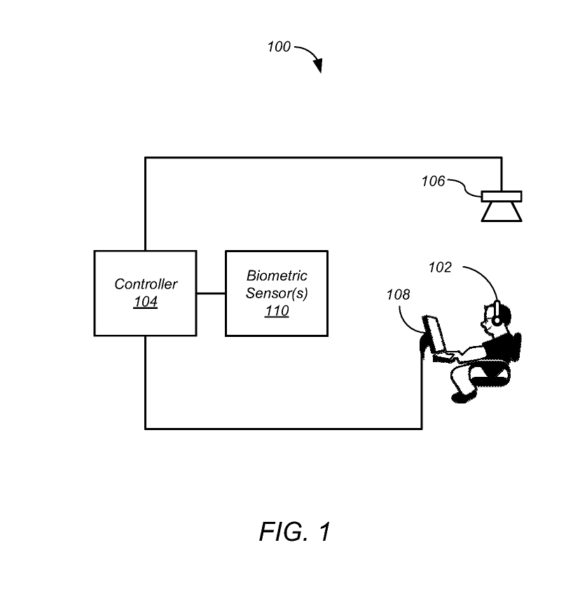

In some embodiments, audio is used to alter the respiratory rate of an individual. FIG. 1 shows elements of an audio wellness system 100 for one user according to one embodiment. Although in the described embodiment elements of the audio wellness system 100 are presented in one arrangement, other embodiments may feature other arrangements. For example, elements of the audio wellness system 100 may be implemented in hardware, software, or combinations thereof. As another example, various elements of the audio wellness system 100 may be implemented as one or more digital signal processors.

Referring to FIG. 1, the audio wellness system 100 may include one or more of a headset 102, one or more controllers 104, one or more speakers 106, and a computer 108. The speakers 106 may be disposed overhead, incorporated in the headset 102, located elsewhere, or any combination thereof. The headset 102 may be any headset. For example, the headset 102 may be wired, wireless, monaural, binaural, and so on. Each speaker 106 and/or headset 102 is in communication with the controller 104. The headset 102 may communicate with the controller 104 through the computer 108. The controller 104 may be implemented as a processor or the like. Some or all of the functions of the controller 104 may be incorporated within the computer 108 and/or the headset 102.

The audio wellness system 100 may include one or more biometric sensors 110. The biometric sensors 110 may be disposed in the workspace of the listeners. For example the biometric sensors 110 may be incorporated in the computer 108, located elsewhere in the workspace, and the like. The biometric sensors 110 may be implemented as wearable sensors. For example the wearable sensors may be incorporated in headsets 102, bracelets, garments, clip-on devices, and the like. Any biometric sensor may be used. For example, the biometric sensors 110 may include microphones and other acoustic sensors, cameras and other light sensors, heat sensors, pressure sensors, tension sensors, inertial sensors, electricity sensors, magnetism sensors, gas sensors, moisture sensors and the like. Any biometrics may be used. For example, the biometrics measured by the biometric sensors 110 may include a gas metric, a moisture metric, a heart rate metric, a sound metric, a galvanic skin response metric, a motion metric, and the like.

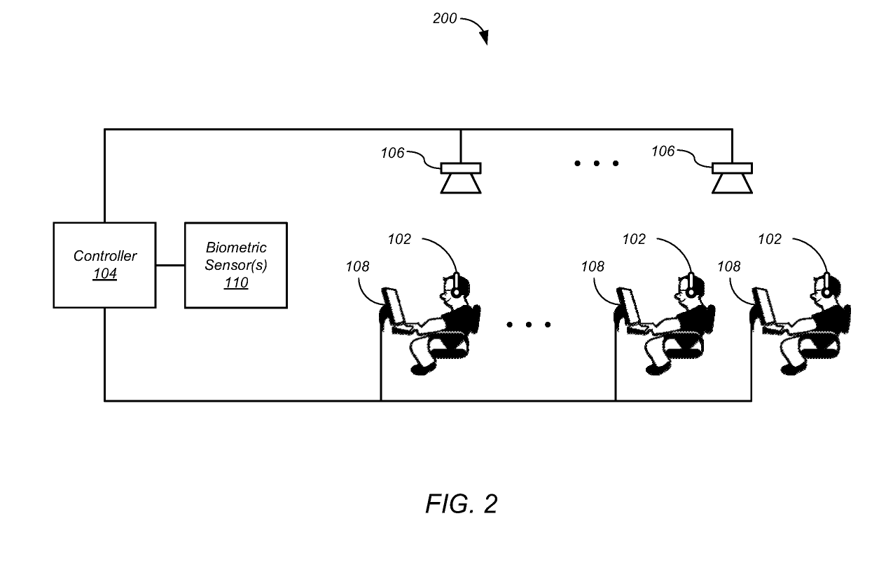

In some embodiments, audio is used to alter the respiratory rates of a group. FIG. 2 shows elements of an audio wellness system 200 for multiple users according to one embodiment. Although in the described embodiment elements of the audio wellness system 200 are presented in one arrangement, other embodiments may feature other arrangements. For example, elements of the audio wellness system 200 may be implemented in hardware, software, or combinations thereof. As another example, various elements of the audio wellness system 200 may be implemented as one or more digital signal processors.

Referring to FIG. 2, the audio wellness system 200 includes one or more speakers 106, one or more headsets 102, a controller 104, and one or more computers 108. The audio wellness system 200 may include one or more biometric sensors 110. Each of these elements may be as described above with reference to FIG. 1.

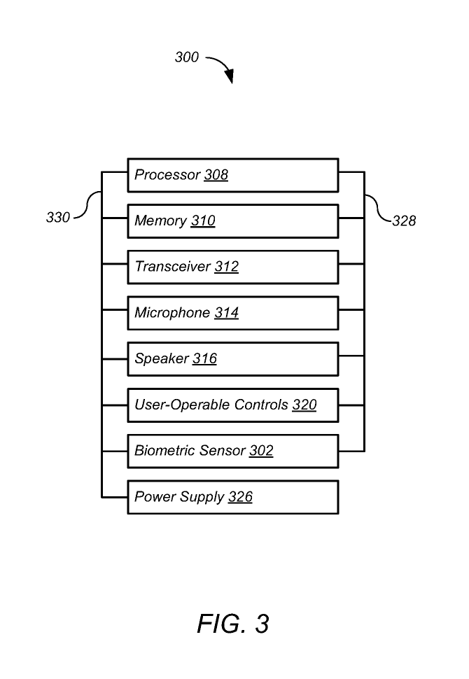

FIG. 3 shows elements of a headset 300 according to one embodiment. The headset 300 may be used as the headsets 102 of FIGS. 1 and 2. Although in the described embodiment elements of the headset 300 are presented in one arrangement, other embodiments may feature other arrangements. For example, elements of the headset 300 may be implemented in hardware, software, or combinations thereof.

Referring to FIG. 3, the headset 300 may include one or more of a biometric sensor 302, a transceiver 312, a processor 308, a memory 310, a microphone 314, a speaker 316, one or more user-operable controls 320, and a power supply 326. The headset 300 may include other elements as well. The elements of headset 300 may receive power from the power supply 326 over one or more power rails 330. Various elements of the headset 300 may be implemented as one or more integrated circuits.

The processor 308 may execute applications stored in the memory 310. The processor 308 may include digital signal processors, analog-to-digital converters, digital-to-analog converters, and the like. The processor 308 may communicate with other elements of the headset 300 over one or more communication busses 328. The transceiver 312 may employ any communication protocol, including wired and wireless communication protocols. The wireless protocols may include Bluetooth, Bluetooth Low-Energy (BLE), Wi-Fi, Digital Enhanced Cordless Telecommunications (DECT), cellular, near-field communications (NFC), and the like. The transceiver 312 may employ multiple communication protocols. The user-operable controls 320 may include buttons, slide switches, capacitive sensors, touch screens, and the like.

The biometric sensor 302 may be implemented as described above for the biometric sensor 110 of FIG. 1. The processor 308 may implement some or all of the functions of the controller 104 of FIG. 1.

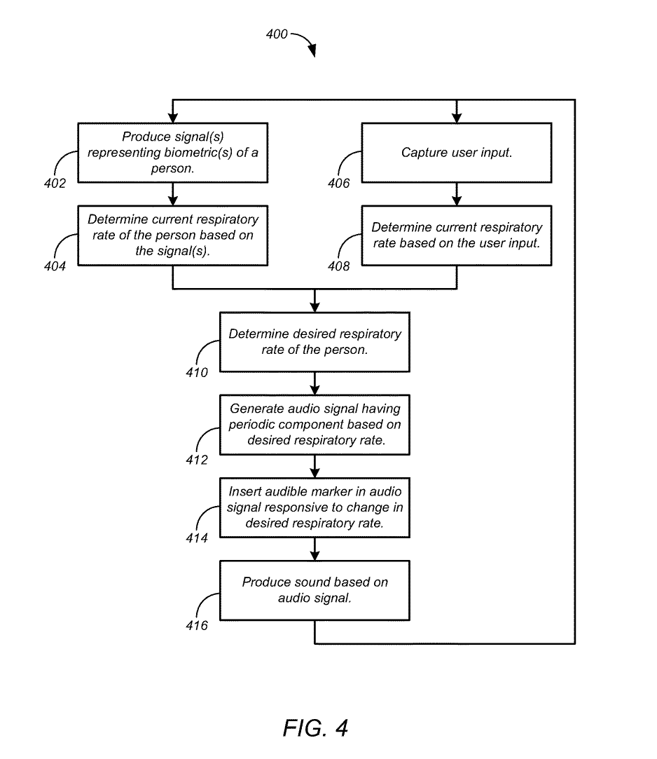

FIG. 4 shows a process 400 for the audio wellness system 100 of FIG. 1 and the audio wellness system 200 of FIG. 2 according to one embodiment. Although in the described embodiments the elements of process 400 are presented in one arrangement, other embodiments may feature other arrangements. For example, in various embodiments, some or all of the elements of process 400 can be executed in a different order, concurrently, and the like. Also some elements of process 400 may not be performed, and may not be executed immediately after each other. In addition, some or all of the elements of process 400 can be performed automatically, that is, without human intervention.

Referring to FIG. 4, at 402, in some embodiments one or more biometric sensors 110 may produce one or more signals representing one or more biometrics of a person. At 404, the controller 104 may determine the current respiratory rate of the person based on the signal(s). At 406, in some embodiments, a user interface may capture user input. For example, the user may provide the input to computer 108. The user may click a mouse button once for each inhalation for example. At 408, the controller 104 may determine the current respiratory rate of the person based on the user input. In some embodiments, other techniques may be used to determine the current respiratory rate of the person.

At 410, the controller 104 may determine the desired respiratory rate. The controller 104 may determine the desired respiratory rate based on the current respiratory rate. At 412, the controller 104 may generate an audio signal having a periodic component, where the rate of the periodic component is based on the desired respiratory rate.

In some embodiments, the current respiratory rate of the person may not be determined. The person's respiratory rate may not be known, for example when the audio wellness system 100 employs loudspeakers 106 in a large environment with many users not using biometric sensors 110. In such cases, the desired respiratory rate may be selected independently of the users' actual respiratory rates. For example an ideal target respiratory rate may be selected such as 12 breaths per minute or the like.

At 414, the controller 104 may insert an audible marker in the audio signal responsive to a change in the desired respiratory rate. At 416 the speaker may produce sound based on the audio signal. The process may repeat, for example at 402 or 406.

In some embodiments, the controller 104 may generate the audio signal only responsive to the current respiratory rate being greater than a reference respiratory rate. In that case, the controller 104 may set the desired respiratory rate to be less than the current respiratory rate. In some embodiments, the controller 104 may generate the audio signal only responsive to the current respiratory rate being less than a reference respiratory rate. In that case the controller 104 may set the desired respiratory rate to be greater than the current respiratory rate. In some embodiments, the controller 104 may generate the audio signal only responsive to the current respiratory rate being outside of a desired range. In that case the controller 104 may set the desired respiratory rate to be within the desired range.

Data captured using the techniques described herein may be used for organizational or industry analytics. An organization could, for example, determine that a particular functional group is regularly breathing too rapidly and might benefit from a vacation or retreat. The data could also be used to provide industry benchmarks such as the respiratory rate of the tech industry, the most mindful company, and the like.

Various embodiments of the present disclosure can be implemented in digital electronic circuitry, or in computer hardware, firmware, software, or in combinations thereof. Embodiments of the present disclosure can be implemented in a computer program product tangibly embodied in a computer-readable storage device for execution by a programmable processor. The described processes can be performed by a programmable processor executing a program of instructions to perform functions by operating on input data and generating output. Embodiments of the present disclosure can be implemented in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Suitable processors include, by way of example, both general and special purpose microprocessors. Generally, processors receive instructions and data from a read-only memory and/or a random access memory. Generally, a computer includes one or more mass storage devices for storing data files. Such devices include magnetic disks, such as internal hard disks and removable disks, magneto-optical disks; optical disks, and solid-state disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and optical disks. Any of the foregoing can be supplemented by, or incorporated in, ASICs (application-specific integrated circuits).

A number of implementations have been described. Nevertheless, various modifications may be made without departing from the scope of the disclosure. Accordingly, other implementations are within the scope of the following claims.

What is claimed is:

1. An apparatus comprising:
a controller configured to
i) determine a desired respiratory rate of a person, and
ii) generate an audio signal having a periodic component, wherein a rate of the periodic component is based on the desired respiratory rate; and
a speaker configured to produce sound based on the audio signal.

2. The apparatus of claim 1, wherein:
the controller is further configured to determine a current respiratory rate of the person.

3. The apparatus of claim 2, wherein:
the controller is further configured to determine the desired respiratory rate based on the current respiratory rate.

4. The apparatus of claim 3, further comprising:
a sensor configured to produce a signal, wherein the signal represents a biometric of the person;
wherein the controller is further configured to determine the current respiratory rate of the person based on the signal.

5. The apparatus of claim 1, wherein:
the sensor is wearable.

6. The apparatus of claim 3, further comprising:
a user interface configured to capture user input of the person;
wherein the controller is further configured to determine the current respiratory rate of the person based on the user input.

7. The apparatus of claim 3, wherein:
the controller is further configured to
iii) determine a current respiratory rate of a further person, and
iv) determine the desired respiratory rate based on the current respiratory rate of the person and the current respiratory rate of the further person.

8. A method comprising:
determining a desired respiratory rate of a person, and
generating a sound having a periodic component, wherein a rate of the periodic component is based on the desired respiratory rate.

9. The method of claim 8, further comprising:
determining a current respiratory rate of the person.

10. The method of claim 9, further comprising:
determining the desired respiratory rate based on the current respiratory rate.

11. The method of claim 10, further comprising:
determining a biometric of the person; and
determining the current respiratory rate of the person based on the biometric.

12. The method of claim 10, further comprising:
capturing user input of the person; and
determining the current respiratory rate of the person based on the user input.

13. The method of claim 10, further comprising:
determining a current respiratory rate of a further person, and
determining the desired respiratory rate based on the current respiratory rate of the person and the current respiratory rate of the further person.

14. Computer-readable media embodying instructions executable by a computer to perform functions comprising:
determining a desired respiratory rate of a person, and
generating a sound having a periodic component, responsive to determining the current respiratory rate, wherein a rate of the periodic component is based on the desired respiratory rate.

15. The computer-readable media of claim 14, wherein the functions further comprise:
determining a current respiratory rate of the person.

16. The computer-readable media of claim 15, wherein the functions further comprise:
determining the desired respiratory rate based on the current respiratory rate.

17. The computer-readable media of claim 16, wherein the functions further comprise:
receiving a biometric of the person; and
determining the current respiratory rate of the person based on the biometric.

18. The computer-readable media of claim 16, wherein the functions further comprise:
capturing user input of the person; and
determining the current respiratory rate of the person based on the user input.

19. The computer-readable media of claim 16, wherein the functions further comprise:
determining a current respiratory rate of a further person, and
determining the desired respiratory rate based on the current respiratory rate of the person and the current respiratory rate of the further person.
